(19) United States
(12) Patent Application Publication
(10) Pub. No.: US 2009/0226889 A1
(43) Pub. Date: Sep. 10, 2009

(54) METHODS AND COMPOSITIONS FOR DETECTING CNS VIRUSES

(76) Inventors: Fan Chen, Fullerton, CA (US); Lilly I. Kong, Covina, CA (US); Ming-Chou Lee, Mission Viejo, CA (US); Jules Chen, Walnut, CA (US); Michelle M. Tabb, Santa Ana, CA (US); Michael Aye, Fountain Valley, CA (US)

Correspondence Address: FOLEY & LARDNER LLP, P.O. BOX 80278, SAN DIEGO, CA, 92138-0278, US

(21) Appl. No.: 12/014,063

(22) Filed: Jan. 14, 2008

(51) Int. Cl. C12Q 1/70

(52) U.S. Cl. 435/5

(57) ABSTRACT

The present invention generally relates to a molecular test of enterovirus, herpes simplex virus-1 and -2, and/or Varicella-Zoster virus, in order to identify patients with a viral infection, in particular a viral infection of the central nervous system. Accordingly methods and compositions are disclosed to determine the presence or absence of a viral pathogen in a biological sample comprising, wherein the target nucleic acids comprise the 5′ UTR of the enterovirus genome, UL29 of herpes simplex virus and gene 36 of Varicella-Zoster virus.

### FIELD OF THE INVENTION

The present invention relates generally to the field of pathogen detection. In particular, the present invention relates to methods of detecting enterovirus, herpes simplex virus, and Varicella-Zoster viruses in a biological sample.

### BACKGROUND OF THE INVENTION

The following description is provided to assist the understanding of the reader. None of the information provided or references cited is admitted to be prior art to the present invention.

Central nervous system viral infection includes viral encephalitis, aseptic meningitis, meningoencephalitis and myelitis. CNS viral infection is a very serious clinical condition with high mortality rate and poor clinical outcome. There are more than 65,000 CNS viral infections reported annually in the United States. Over 100 viruses are known to cause CNS infections. Among them, Enterovirus (EV), Herpes Simplex Virus type 1 and type 2 (HSV-1 and -2) are the most common and prevalent pathogens causing this disease. In the past decade, Varicella-Zoster Virus (VZV) has also been recognized as an important pathogen causing CNS infection.

EV's are small RNA viruses (plus sense) of the Picoravirus family. At least sixty-four unique serotypes of EV had been identified and classified, which are sub-grouped into Poliovirus (PV), Coxsackievirus A (CVA) and Coxsackievirus B (CVB), Echovirus (EC), and Enterovirus (EV) 68-81. EV causes oral-fecal transmitted diseases, which occur predominantly as summer and early fall illness. EV infection is very common and mostly manifests as a nonspecific febrile illness. In immuno-compromised individuals, however, it can pass the blood-brain-barrier and cause CNS infection. Due to the large number of serotypes, the clinical presentations vary from various degrees of encephalitis, aseptic meningitis, and myelitis, or mixture of them.

HSV-1 and -2 (DNA viruses) are members of the group alpha herpes viral family. HSV-1 and -2 cause common infections of skin, eye, mouth, and genital tissues. Although more than 50% of the general population have lifetime exposure to HSV infections, invasion of HSV into the CNS is uncommon and occurs predominantly in immuno-compromised individuals. Clinically, CNS infections by HSV-1 and -2 present acute sporadic focal encephalitis. Without treatment, the mortality rate can be as high as 50%, and permanent brain damage often occurs.

VZV (HHV3) is a DNA virus, and is the third member in the group alpha herpes family. Like HSV, VZV is neurotrophic. Primary infection of VZV causes chickenpox. Since the early 1990s, increasing cases of VZV-related CNS viral infections have been reported. Reactivation of VZV in peripheral neural tissues in immuno-compromised individuals is believed to be the cause of CNS infection.

### SUMMARY OF THE INVENTION

The present invention generally relates to a molecular test of EV, HSV-1/-2, and/or VZV to identify patients with a viral infection. In one aspect, the present invention provides a method for identifying the presence or absence of a viral pathogen in a biological sample, comprising assaying for one or both of a HSV UL29 nucleic acid or a fragment thereof and a VZV gene 36 nucleic acid or fragment thereof, wherein the presence of one or both of the nucleic acids or fragments indicates that the biological sample contains the viral pathogen associated with said nucleic acids or fragments. In one embodiment, the methods further comprise assaying for an enterovirus 5′ UTR nucleic acid or a fragment thereof, wherein the presence of the enterovirus 5′ UTR nucleic acid or fragment indicates that the biological sample contains enterovirus.

The target nucleic acids described herein may be detected singly or in any combination, for example, in a multiplex amplification reaction. In one embodiment, the enterovirus 5′ UTR nucleic acid or fragment and the HSV UL29 nucleic acid or fragment are detected in a multiplex amplification reaction. In another embodiment, the enterovirus 5′ UTR nucleic acid or fragment the HSV UL29 nucleic acid or fragment, and the VZV gene 36 nucleic acid or fragment are detected in a multiplex amplification reaction.

In one embodiment, the step of assaying comprises (a) contacting a biological sample with one or more primers suitable for amplifying an enterovirus 5′ UTR nucleic acid or a fragment thereof; one or more primers suitable for amplifying an HSV UL29 nucleic acid or a fragment thereof; and one or more primers suitable for amplifying a VZV gene 36 nucleic acid or a fragment thereof; (b) performing a multiplex amplification reaction comprising the primer pairs of step (a) under conditions suitable to produce a first reaction product when the enterovirus 5′ UTR nucleic acid is present in said sample, a second reaction product when HSV UL29 nucleic acid is present in said sample; a third reaction product suitable for amplifying the VZV gene 36 nucleic acid is present in said sample; and (c) detecting the presence of one or more of the first, second, or third reaction products.

In one embodiment, each of the first, second, and/or third reaction products comprises at least 15, at least 20, at least 30, at least 40, at least 50, at least 75, at least 100 contiguous nucleotides of the respective target sequences. In one embodiment, the first reaction product has at least 30 contiguous nucleotides from the sequence of SEQ ID NO: 1. In one embodiment, the second reaction product has at least 30 contiguous nucleotides from the sequence of SEQ ID NO: 2. In one embodiment, the third reaction product has at least 30 contiguous nucleotides from the sequence of SEQ ID NO: 3.

In one aspect, the present invention provides nucleic acid primers, probes and bi-functional molecules for the amplification and detection of the target nucleic acids described herein. In one embodiment, at least one primer of the first primer pair comprises a sequence selected from the group consisting of: SEQ ID NOS: 4, 8, and complements thereof. In one embodiment, at least one primer of the second primer pair comprises a sequence selected from the group consisting of: SEQ ID NOS: 9-10, and complements thereof. In one embodiment, at least one primer of the third primer pair comprises a sequence selected from the group consisting of: SEQ ID NOS: 13-14, and complements thereof.

In one embodiment, one or more of the first, second, or third reaction products are detected using a probe comprising a fluorescent label. In one embodiment, the probe is an oligonucleotide complementary to the target sequence. In another embodiment, a probe and one of the primers of the first, second, and/or third primer pairs are part of a bi-functional molecule, i.e., a Scorpion™. In particular embodiments, the bi-functional molecule(s) have a sequence according to: SEQ ID NOS: 6-7, 11-12, and/or 15.

In one embodiment, the step of detecting comprises performing an invasive cleavage technique on one or more of the nucleic acids or fragments specified in (a), (b) or (c). In some embodiments, the amplification and detection of the target nucleic acids comprise real-time PCR.

In another aspect, the present invention provides a method of diagnosing a subject for infection with a viral pathogen, comprising assaying a biological sample from the subject for one or both of a HSV UL29 nucleic acid or a fragment thereof and a VZV gene 36 nucleic acid or fragment thereof; wherein the presence of one or both of the nucleic acids or fragments indicates that the individual is affected with the viral pathogen associated with said nucleic acids or fragments. In one embodiment, the methods further comprise assaying for an enterovirus 5′ UTR nucleic acid or a fragment thereof, wherein the presence of the enterovirus 5′ UTR nucleic acid or fragment indicates that the biological sample contains enterovirus.

In another aspect, the present invention provides kits comprising (a) a primer pair suitable for amplifying an enterovirus 5′ UTR nucleic acid or fragment thereof and a probe capable of specifically hybridizing to the enterovirus 5′ UTR nucleic acid; (b) a primer pair one or more primers suitable for amplifying an HSV UL29 nucleic acid or a fragment thereof and a probe capable of specifically hybridizing to the HSV UL29 nucleic acid; and/or (c) a primer pair suitable for amplifying a VZV gene 36 nucleic acid or a fragment thereof and a probe capable of specifically hybridizing to the VZV gene 36 nucleic acid.

### DETAILED DESCRIPTION

The present invention provides methods for detecting viral pathogens involved in CNS infections. For encephalitis, urgent management and immediate treatment are required. Accurate and early diagnosis are essential, so that anti-viral therapy (e.g. Acyclovir™ or Pleconaril™) may be prescribed, if appropriate. The benefits of a molecular diagnostic test for CNS infections include a reduced hospital stay, reduced antimicrobial/antibiotic exposure, and reduced hospital cost. Quantitative PCR tests of CSF may be useful in assessing the severity of CNS disease and for monitoring antiviral therapy.

Units, prefixes, and symbols may be denoted in their accepted SI form. Unless otherwise indicated, nucleic acids are written left to right in 5′ to 3′ orientation. Nucleotides, may be referred to by their commonly accepted single-letter codes.

As used herein, unless otherwise stated, the singular forms “a,” “an,” and “the” include plural reference. Thus, for example, a reference to “an oligonucleotide” includes a plurality of oligonucleotide molecules, and a reference to “a nucleic acid” is a reference to one or more nucleic acids.

As used herein, “about” means plus or minus 10% unless otherwise indicated.

The terms “amplification” or “amplify” as used herein includes methods for copying a target nucleic acid, thereby increasing the number of copies of a selected nucleic acid sequence. Amplification may be exponential or linear. A target nucleic acid may be either DNA or RNA. The sequences amplified in this manner form an “amplicon.” While the exemplary methods described hereinafter relate to amplification using the polymerase chain reaction (PCR), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Innis et al., Eds., Academic Press, San Diego, Calif. 1990, pp 13-20; Wharam, et al., Nucleic Acids Res. 2001 Jun. 1; 29(11):E54-E54; Hafner, et al., Biotechiques 2001 April; 30(4):852-6, 858, 860; Zhong, et al., Biotechiques 2001 April; 30(4):852-6, 858, 860.

The term “complement” “complementary” or “complementarity” as used herein with reference to polynucleotides (i.e., a sequence of nucleotides such as an oligonucleotide or a target nucleic acid) refers to standard Watson/Crick pairing rules. The complement of a nucleic acid sequence such that the 5′ end of one sequence is paired with the 3′ end of the other, is in “antiparallel association.” For example, the sequence “5′-A-G-T-3′” is complementary to the sequence “3′-T-C-A-5′.” Certain bases not commonly found in natural nucleic acids may be included in the nucleic acids described herein; these include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be a sequence of RNA complementary to the DNA sequence or its complement sequence, and can also be a cDNA. The term “substantially complementary” as used herein means that two sequences specifically hybridize (defined below). The skilled artisan will understand that substantially complementary sequences need not hybridize along their entire length.

As used herein, the term “detecting” used in context of detecting a signal from a detectable label to indicate the presence of a target nucleic acid in the sample does not require the method to provide 100% sensitivity and/or 100% specificity. As is well known, “sensitivity” is the probability that a test is positive, given that the subject has a target nucleic acid sequence, while “specificity” is the probability that a test is negative, given that the subject does not have the target nucleic acid sequence. A sensitivity of at least 50% is preferred, although sensitivities of at least 60%, at least 70%, at least 80%, at least 90% and at least 99% are clearly more preferred. A specificity of at least 50% is preferred, although sensitivities of at least 60%, at least 70%, at least 80%, at least 90% and at least 99% are clearly more preferred. Detecting also encompasses assays with false positives and false negatives. False negative rates may be 1%, 5%, 10%, 15%, 20% or even higher. False positive rates may be 1%, 5%, 10%, 15%, 20% or even higher.

A “fragment” in the context of a nucleic acid refers to a sequence of contiguous nucleotide residues which are at least about 5 nucleotides, at least about 7 nucleotides, at least about 9 nucleotides, at least about 11 nucleotides, or at least about 17 nucleotides. The fragment is typically less than about 300 nucleotides, less than about 100 nucleotides, less than about 75 nucleotides, less than about 50 nucleotides, or less than 30 nucleotides. In certain embodiments, the fragments can be used in polymerase chain reaction (PCR), various hybridization procedures or microarray procedures to identify or amplify identical or related parts of mRNA or DNA molecules. A fragment or segment may uniquely identify each polynucleotide sequence of the present invention.

“Genomic nucleic acid,” “genomic DNA,” or “genomic RNA” refers to some or all of the DNA from a chromosome. Genomic DNA or RNA may be intact or fragmented (e.g., digested with restriction endonucleases by methods known in the art). In some embodiments, genomic DNA or RNA may include sequence from all or a portion of a single gene or from multiple genes. In contrast, the term “total genomic nucleic acid” is used herein to refer to the full complement of DNA or RNA contained in the genome. Methods of purifying DNA and/or RNA from a variety of samples are well-known in the art.

The term “multiplex PCR” as used herein refers to an assay that provides for simultaneous amplification and detection of two or more products within the same reaction vessel. Each product is primed using a distinct primer pair. A multiplex reaction may further include specific probes for each product, that are detectably labeled with different detectable moieties.

As used herein, the term “oligonucleotide” refers to a short polymer composed of deoxyribonucleotides, ribonucleotides or any combination thereof. Oligonucleotides are generally between about 10, 11, 12, 13, 14 or 15 to about 150 nucleotides (nt) in length, more preferably about 10, 11, 12, 13, 14, or 15 to about 70 nt, and most preferably between about 18 to about 26 nt in length. The single letter code for nucleotides is as described in the U.S. Patent Office Manual of Patent Examining Procedure, section 2422, table 1. In this regard, the nucleotide designation “R” means purine such as guanine or adenine, “Y” means pyrimidine such as cytosine or thymidine (uracil if RNA); and “M” means adenine or cytosine. An oligonucleotide may be used as a primer or as a probe.

As used herein, a “primer” for amplification is an oligonucleotide that is complementary to a target nucleotide sequence and leads to addition of nucleotides to the 3′ end of the primer in the presence of a DNA or RNA polymerase. The 3′ nucleotide of the primer should generally be identical to the target sequence at a corresponding nucleotide position for optimal expression and/or amplification. The term “primer” as used herein includes all forms of primers that may be synthesized including peptide nucleic acid primers, locked nucleic acid primers, phosphorothioate modified primers, labeled primers, and the like. As used herein, a “forward primer” is a primer that is complementary to the anti-sense strand of dsDNA. A “reverse primer” is complementary to the sense-strand of dsDNA.

An oligonucleotide (e.g., a probe or a primer) that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions.

“Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Equations for calculating Tₘ and conditions for nucleic acid hybridization are known in the art.

As used herein, an oligonucleotide is “specific” for a nucleic acid if the oligonucleotide has at least 50% sequence identity with a portion of the nucleic acid when the oligonucleotide and the nucleic acid are aligned. An oligonucleotide that is specific for a nucleic acid is one that, under the appropriate hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity. Sequence identity can be determined using a commercially available computer program with a default setting that employs algorithms well known in the art (e.g., BLAST). As used herein, sequences that have “high sequence identity” have identical nucleotides at least at about 50% of aligned nucleotide positions, preferably at least at about 60% of aligned nucleotide positions, and more preferably at least at about 75% of aligned nucleotide positions.

Oligonucleotides used as primers or probes for specifically amplifying (i.e., amplifying a particular target nucleic acid sequence) or specifically detecting (i.e., detecting a particular target nucleic acid sequence) a target nucleic acid generally are capable of specifically hybridizing to the target nucleic acid.

As used herein, the term “sample” or “test sample” may comprise clinical samples, isolated nucleic acids, or isolated microorganisms. In preferred embodiments, a sample is obtained from a biological source (i.e., a “biological sample”), such as tissue, bodily fluid, or microorganisms collected from a subject. Sample sources include, but are not limited to, sputum (processed or unprocessed), bronchial alveolar lavage (BAL), bronchial wash (BW), blood, bodily fluids, cerebrospinal fluid (CSF), urine, plasma, serum, or tissue (e.g., biopsy material). The term “patient sample” as used herein refers to a sample obtained from a human seeking diagnosis and/or treatment of a disease.

As used herein, the term “Scorpion™ detection system” refers to a method for real-time PCR. This method utilizes a bi-functional molecule (referred to herein as a “Scorpion™”), which contains a PCR primer element covalently linked by a polymerase-blocking group to a probe element. Additionally, each Scorpion™ molecule contains a fluorophore that interacts with a quencher to reduce the background fluorescence. In a particular embodiment, the Scorpion™ molecule comprises, in 5′ to 3′ order, a quencher, a probe region, a fluorophore, a linker region, and a primer region.

The terms “target nucleic acid” or “target sequence” as used herein refer to a sequence which includes a segment of nucleotides of interest to be amplified and detected. Copies of the target sequence which are generated during the amplification reaction are referred to as amplification products, amplimers, or amplicons. Target nucleic acid may be composed of segments of a chromosome, a complete gene with or without intergenic sequence, segments or portions of a gene with or without intergenic sequence, or sequence of nucleic acids which probes or primers are designed. Target nucleic acids may include a wild-type sequence(s), a mutation, deletion or duplication, tandem repeat regions, a gene of interest, a region of a gene of interest or any upstream or downstream region thereof. Target nucleic acids may represent alternative sequences or alleles of a particular gene. Target nucleic acids may be derived from genomic DNA, cDNA, or RNA. As used herein target nucleic acid may be DNA or RNA extracted from a cell or a nucleic acid copied or amplified therefrom.

As used herein “TaqMan® PCR detection system” refers to a method for real time PCR. In this method, a TaqMan® probe which hybridizes to the nucleic acid segment amplified is included in the PCR reaction mix. The TaqMan® probe comprises a donor and a quencher fluorophore on either end of the probe and in close enough proximity to each other so that the fluorescence of the donor is taken up by the quencher. However, when the probe hybridizes to the amplified segment, the 5′-exonuclease activity of the Taq polymerase cleaves the probe thereby allowing the donor fluorophore to emit fluorescence which can be detected.

### METHODS OF THE PRESENT INVENTION

In accordance with the present invention, there are provided methods for identifying a viral infection in a subject. The methods provide for detection of human Enteroviruses (EV), Herpes Simplex viruses types-1 & -2 (HSV-1 & -2) and/or Varicella-Zoster virus (VZV) in biological samples, e.g. specimens of human cerebrospinal fluid (CSF) from subjects with signs and symptoms of meningitis, encephalitis, or meningoencephalitis. Consequently, the methods of the invention, in conjunction with other laboratory results and clinical information, may be used in the diagnosis of EV, HSV-1/-2, and/or VZV infection in subjects with a clinical suspicion of meningitis, encephalitis, or meningoencephalitis. Infections typically comprise a single viral pathogen, as dual-infection of CSF by EV, HSV, and VZV is very unlikely. Triple infection of CSF by EV, HSV, and VZV has not been reported.

In various embodiments of the present invention, oligonucleotide primers and probes are used in the methods described herein to provide the viral pathogen assay. Thus, in certain embodiments, the invention relates to primer sequences that can be used to amplify target nucleic acids EV, HSV-1/-2, and VZV in a multiplex reaction. In particular embodiments, the target nucleic acids include the 5′ UTR for EV, the UL29 gene for HSV-1/-2, and gene 36 for VZV.

In certain embodiments, the methods and kits utilize Scorpion™ technology and a reverse primer for the real-time PCR amplification and detection of the target nucleic acids. Scorpion™ technology utilizes a bi-functional molecule containing a PCR primer element covalently linked by a polymerase-blocking group to a probe element. Each molecule contains a quencher that interacts with a fluorophore. The target is amplified by the reverse primer and the primer portion of the Scorpion™ specific for that target. A fluorescent signal is generated after the separation of the fluorophore from the quencher as a result of the binding of the probe element of the Scorpion™ to the extended DNA fragment.

In some embodiments, the assay further includes an internal control (IC) to verify adequate processing of the target viruses and reaction setup and to monitor the presence of inhibition in the RT-PCR assay to avoid false negative results.

In one embodiment, the method provides for the extraction of nucleic acids from the subject's CSF specimens for use as the testing template followed by one-step RT-PCR using reverse transcription to convert target RNA to cDNA followed by the simultaneous amplification and detection of the target template.

In one aspect, the invention relates to one or more substantially purified oligonucleotides having sequences selected from the primers and Scorpions™ shown in Table 1.

TABLE 1

Exemplary Primer and Scorpion ™ Sequences
for Pathogen Detection

| | | |
|---|---|---|
| Primer Name | Sequence (5′ to 3′) | SEQ ID NO: |

| | | |
|---|---|---|
| EV Forward | TCCGGCCCCTGAATGC | SEQ ID NO: 4 |
| EV Dx2B | Quencher- | SEQ ID NO: 5 |
| Scorpion ™ | AGCGCGCACCCAAAGTAGTCGGTTCCGCGCGCT- | |
| | dye-TCCGGCCCCTGAATGC | |
| EV Dx2I-mpn | Quencher- | SEQ ID NO: 6 |
| Scorpion ™ | AGCGGCACGGACACCCAAAGTAGTCGGCCGCT- | |
| | dye-TCCGGCCCCTGAATGC | |
| EV DX2-mpn | Quencher- | SEQ ID NO: 7 |
| Scorpion ™ | AGCGGGCCAAAGTAGTCGGTTCCGCCCGCT-dye- | |
| | TCCGGCCCCTGAATGC | |
| EV Reverse | CAATTGTCACCATAAGCAGCCA | SEQ ID NO: 8 |
| HSV Forward | GGTCCGAGGAGGATGTCC | SEQ ID NO: 9 |
| HSV Reverse | CGTCCGAGGCCGCCAA | SEQ ID NO: 10 |
| HSV Dx2E | Quencher-AGCGCTGAGCGCCTACCAGAAGCGCT- | SEQ ID NO: 11 |
| Scorpion ™ | dye-GGTCCGAGGAGGATGTCC | |
| HSV Dx2C | Quencher- | SEQ ID NO: 12 |
| Scorpion ™ | AGGCGCCTACCAGAAGCCCGACAAGCGCCT-dye- | |
| | GGTCCGAGGAGGATGTCC | |
| VZV Forward | GTTATTGTTTACGCTTCCCGCTGAA | SEQ ID NO: 13 |
| VZV Reverse | GCCCGTTTGCTTACTCTGGATAA | SEQ ID NO: 14 |
| VZV Dx2 | Quencher- | SEQ ID NO: 15 |
| Scorpion ™ | AGCGGAGTGAAACGGTACAAACTCCGCT-dye- | |
| | GTTATTGTTTACGCTTCCCGCTGAA | |
| IC Forward Primer | ATTCGCCCTTTGTTTCGACCTA | SEQ ID NO: 16 |
| IC Dx2 | Quencher- TGCGAACTGGCAAGCT-dye- | SEQ ID NO: 17 |
| Scorpion ™ | ATTCGCCCTTTGTTTCGACCTA | |
| IC Reverse | CCGACGACTGACGAGCAA | SEQ ID NO: 18 |

Sample Preparation. Specimens from which target nucleic acids can be detected and quantified with the methods of the present invention are from sterile and/or non-sterile sites. Sterile sites from which specimens can be taken are body fluids such as blood, urine, cerebrospinal fluid (CSF), synovial fluid, pleural fluid, pericardial fluid, intraocular fluid, tissue biopsies or endotracheal aspirates. Non-sterile sites from which specimens can be taken are e.g., sputum, stool, swabs from e.g. skin, inguinal, nasal and/or throat. Preferably, specimens from CSF are used in the present invention. Specimens for CNS virus detection may also comprise viral cultures.

The nucleic acid (DNA and/or RNA) may be isolated from the sample according to any methods well known to those of skill in the art. If necessary, the sample may be collected or concentrated by centrifugation and the like. The cells of the sample may be subjected to lysis, such as by treatments with enzymes, heat surfactants, ultrasonication or combinations thereof. The lysis treatment is performed in order to obtain a sufficient amount of DNA derived from the viral pathogens, if present in the sample, to detect using polymerase chain reaction.

Various methods of DNA extraction are suitable for isolating the DNA. Suitable methods include phenol and chloroform extraction. See Maniatis et al., Molecular Cloning, A Laboratory Manual, 2d, Cold Spring Harbor Laboratory Press, page 16.54 (1989). Numerous commercial kits also yield suitable DNA including, but not limited to, QIAamp™ mini blood kit, QIAamp™ mini viral RNA kit, Agencourt Genfind™, Roche Cobas® Roche MagNA Pure® or phenol:chloroform extraction using Eppendorf Phase Lock Gels®.

In one embodiment, a nucleic acid isolation step is used that isolates both RNA and DNA in one reaction. In an alternate embodiment, RNA and DNA may be isolated independently and then combined for use in the methods of the invention. In yet another alternate embodiment, when only one type of nucleic acid is required to be isolated (such as when all the disease agents and secondary disease agents of interest have the same type of nucleic acid genome), nucleic acid isolation methods that isolate only RNA or DNA may be used. A variety of techniques and protocols are known in the art for simultaneous RNA and DNA isolation and the separate isolation of each and such techniques and protocols may be used. The nucleic acid isolation described may be used to isolate nucleic acid from a variety of patient samples or sources. The types of patient samples/sources include, but are not limited to, CSF, nasal/pharyngeal swabs, saliva, sputum, serum, whole blood and stool.

In one embodiment, a dual RNA/DNA isolation method is used employing a trizol based reagent for initial isolation of RNA and DNA from patient samples. Upon contact with patient samples, the phenol and high salt reagents in the trizol effectively inactivate any disease agent or secondary disease agent that may be present in the patient sample. In order to allow for the dual isolation of RNA and DNA in the same phase with a single step, the pH of the trizol solution may be adjusted towards neutral (instead of acidic). After the RNA and DNA are isolated from the patient samples, a silica based column may be used to further isolate the RNA and DNA. The use of silica based columns allows for wash steps to be performed quickly and efficiently while minimizing the possibility of contamination. The wash steps may be used to remove PCR and RT-PCR inhibitors. The column method for nucleic acid purification is advantageous as it can be used with different types of patient samples and the spin and wash steps effectively remove PCR or RT-PCR inhibitors. In one embodiment, the nucleic isolation is carried out using the dual RNA/DNA isolation kit provided by QIAamp® Viral RNA Mini Spin Kit (Qiagen, Valencia, Calif.).

Amplification of Nucleic Acids. Nucleic acid samples or target nucleic acids may be amplified by various methods known to the skilled artisan. Preferably, PCR is used to amplify nucleic acids of interest. Briefly, in PCR, two primer sequences are prepared that are complementary to regions on opposite complementary strands of the marker sequence. An excess of deoxynucleotide triphosphates are added to a reaction mixture along with a DNA polymerase, e.g., Taq polymerase.

In one embodiment, the target nucleic acids are amplified in a multiplex amplification reaction. A variety of multiplex amplification strategies are known in the art and may be used with the methods of the invention. The multiplex amplification strategy may use PCR, RT-PCR or a combination thereof depending on the type of nucleic acid contained in the disease agent(s). For example, if an RNA genome is present, RT-PCR may be utilized. The PCR enzyme may be an enzyme with both a reverse transcription and polymerase function. Furthermore, the PCR enzyme may be capable of “hot start” reactions as is known in the art.

If the target sequence is present in a sample, the primers will bind to the sequence and the polymerase will cause the primers to be extended along the target sequence by adding on nucleotides. By raising and lowering the temperature of the reaction mixture, the extended primers will dissociate from the target nucleic acid to form reaction products, excess primers will bind to the target nucleic acid and to the reaction products and the process is repeated, thereby generating amplification products. Cycling parameters can be varied, depending on the length of the amplification products to be extended. An internal positive amplification control (IC) can be included in the sample, utilizing oligonucleotide primers and/or probes.

In a suitable embodiment, PCR is performed using a Scorpion™ primer/probe combination. Scorpion™ probes, as used in the present invention comprise a 3′ primer with a 5′ extended probe tail comprising a hairpin structure which possesses a fluorophore/quencher pair. The probe tail is “protected” from replication in the 5′ to 3′ direction by the inclusion of hexethlyene glycol (HEG) which blocks the polymerase from replicating the probe. During the first round of amplification the 3′ target-specific primer anneals to the target and is extended such that the Scorpion™ is now incorporated into the newly synthesized strand, which possesses a newly synthesized target region for the 5′ probe. During the next round of denaturation and annealing, the probe region of the Scorpion™ hairpin loop will hybridize to the target, thus separating the fluorophore and quencher and creating a measurable signal. Such probes are described in whitcombe et al., Nature Biotech 17: 804-807 (1999).

### Target Nucleic Acids and Primers

The methods of the present invention relate to the detection of viral pathogens in biological samples. Detection may include EV, HSV-1/-2, and/or VZV. For example, the pathogens may be detected singly or in any combination in a multiplex amplification reaction. In one embodiment, the target nucleic acid for EV is a consensus sequence identified from two or more EV serotypes. Consequently, primers designed to be complementary to the consensus sequence will amplify multiple EV serotypes, if present in the sample.

A comprehensive study of EV surveillance in the United States from 1970-2005 (CDC) showed that variable serotypes collectively accounted for about 23.38% of all reported EV CNS infection cases. Among them, variable serotype EC11 was ranked number two, and EC11 alone accounted for 11.4% of all reported EV CNS infection cases. Therefore, in particular embodiments, the target nucleic acid for EV may comprise a consensus sequence capable of detecting the most common disease-causing enterovirus serotypes.

In one embodiment, the target nucleic acid for EV is the 5′ UTR of the EV genome. In particular embodiments, primers are designed based on a consensus sequence of two or more serotypes so that multiple serotypes may be detected with one primer pair. For example, a sequence according to SEQ ID NO: 1 is a consensus sequence of at least 64 serotypes of EV. Primers may be complementary to a portion of SEQ ID NO: 1 in order to amplify a portion thereof from multiple EV serotypes in a sample, if present. For example, primers according to SEQ ID NOS: 4 and 8 may be used to amplify the 5′ UTR of EV. In one embodiment, a Scorpion™ primer/probe, e.g. according to SEQ ID NOS: 6 or 7 may be used.

TABLE 2
EV 5′ UTR Consensus Sequence (SEQ ID NO: 1)

TTGGTAGTCCTCCGGCCCCTGAATGCGGCTAATCCTAACTGCGGAGCACG
TGCCCACAAACCAGTGGGTAGTGTGTCGTAACGGGCAACTCTGCAGCGGA
ACCGACTACTTTGGGTGTCCGTGTTTCCTTTTATTCTTATACTGGCTGCT
TATGGTGACAATTGAGAGATTGTTACCATATAGCTATTGGATTGGCCATC
CGGTGACTAACAGAGCTATTATATACCTGTTT

In one embodiment, the target nucleic acid for HSV-1/-2 is the UL29 target of the HSV genome. The nucleotide sequence of UL29 is shown in Table 3 (SEQ ID NO: 2). Primers may be complementary to a portion of SEQ ID NO: 2 in order to amplify the target from HSV-1 and/or HSV-2 in a sample, if present. For example, primers according to SEQ ID NOS: 9 and 10 may be used to amplify the UL29 target nucleic acid of HSV-1/-2. In one embodiment, a Scorpion™ primer/probe, e.g. according to SEQ ID NOS: 11 or 12 may be used.

TABLE 3
HSV UL29 Sequence (SEQ ID NO: 2)

CCGCGTGGAACTGCTTCAGCAGAAAGCCCAGCGGTCCGAGGAGGATGTCC
ACGCGCTTGTCGGGCTTCTGGTAGGCGCTCTGGAGGCTGGCGACCCGCGC
CTTGGCGGCCTCGGACGCGT

In one embodiment, the target nucleic acid for VZV is Gene 36 of the VZV genome. The nucleotide sequence of Gene 36 is shown in Table 4 (SEQ ID NO: 3). Primers may be complementary to a portion of SEQ ID NO: 3 in order to amplify the target from VZV in a sample, if present. For example, primers according to SEQ ID NOS: 13 and 14 may be used to amplify the gene 36 of VZV. In one embodiment, a Scorpion™ primer/probe, e.g. according to SEQ ID NO: 15 may be used.

TABLE 4
VZV Gene 36 Sequence (SEQ ID NO: 3)

TTTCCCTTGTCCAGATACTTAGTGGGAGATATGTCCCCAGCGGCGCTTCC
TGGGTTATTGTTTACGCTTCCGCTGAACCCCCCGGGACCAACTTGGTAGT
TTGTACCGTTTCACTCCCCAGTCATTTATCCAGAGTAAGCAAACGGGCCA
GACCGGGAGAAACGGTTAATCTGCCGTTT

### Detection of Amplified Nucleic Acids

Amplification of nucleic acids can be detected by any of a number of methods well-known in the art such as gel electrophoresis, column chromatography, hybridization with a probe, sequencing, melting curve analysis, or “real-time” detection.

In one approach, sequences from two or more fragments of interest are amplified in the same reaction vessel (i.e. “multiplex PCR”). Detection can take place by measuring the end-point of the reaction or in “real time.” For real-time detection, primers and/or probes may be detectably labeled to allow differences in fluorescence when the primers become incorporated or when the probes are hybridized, for example, and amplified in an instrument capable of monitoring the change in fluorescence during the reaction. Real-time detection methods for nucleic acid amplification are well known and include, for example, the TaqMan® system, the Scorpion™ bi-functional molecule, and the use of intercalating dyes for double stranded nucleic acid.

In end-point detection, the amplicon(s) could be detected by first size-separating the amplicons, then detecting the size-separated amplicons. The separation of amplicons of different sizes can be accomplished by, for example, gel electrophoresis, column chromatography, or capillary electrophoresis. These and other separation methods are well-known in the art. In one example, amplicons of about 10 to about 150 base pairs whose sizes differ by 10 or more base pairs can be separated, for example, on a 4% to 5% agarose gel (a 2% to 3% agarose gel for about 150 to about 300 base pair amplicons), or a 6% to 10% polyacrylamide gel. The separated nucleic acids can then be stained with a dye such as ethidium bromide and the size of the resulting stained band or bands can be compared to a standard DNA ladder.

In another embodiment, two or more fragments of interest are amplified in separate reaction vessels. If the amplification is specific, that is, one primer pair amplifies for one fragment of interest but not the other, detection of amplification is sufficient to distinguish between the two types—size separation would not be required.

In some embodiments, amplified nucleic acids are detected by hybridization with a specific probe. Probe oligonucleotides, complementary to a portion of the amplified target sequence may be used to detect amplified fragments. Hybridization may be detected in real time or in non-real time. Amplified nucleic acids for each of the target sequences may be detected simultaneously (i.e., in the same reaction vessel) or individually (i.e., in separate reaction vessels). In preferred embodiments, the amplified DNA is detected simultaneously, using two or more distinguishably-labeled, gene-specific oligonucleotide probes, one which hybridizes to the first target sequence and one which hybridizes to the second target sequence.

The probe may be detectably labeled by methods known in the art. Useful labels include, e.g., fluorescent dyes (e.g., Cy5®, Cy3®, FITC, rhodamine, lanthamide phosphors, Texas red, FAM, JOE, Cal Fluor Red 610-®, Quasar 670®), ³²P, ³⁵S, ³H, ¹⁴C, ¹²⁵I, ¹³¹I, electron-dense reagents (e.g., gold), enzymes, e.g., as commonly used in an ELISA (e.g., horseradish peroxidase, beta-galactosidase, luciferase, alkaline phosphatase), colorimetric labels (e.g., colloidal gold), magnetic labels (e.g., Dynabeads™), biotin, dioxigenin, or haptens and proteins for which antisera or monoclonal antibodies are available. Other labels include ligands or oligonucleotides capable of forming a complex with the corresponding receptor or oligonucleotide complement, respectively. The label can be directly incorporated into the nucleic acid to be detected, or it can be attached to a probe (e.g., an oligonucleotide) or antibody that hybridizes or binds to the nucleic acid to be detected.

One general method for real time PCR uses fluorescent probes such as the TaqMan® probes, molecular beacons, and Scorpions™. Real-time PCR quantitates the initial amount of the template with more specificity, sensitivity and reproducibility, than other forms of quantitative PCR, which detect the amount of final amplified product. Real-time PCR does not detect the size of the amplicon. The probes employed in Scorpion™ and TaqMan® technologies are based on the principle of fluorescence quenching and involve a donor fluorophore and a quenching moiety.

In a preferred embodiment, the detectable label is a fluorophore. The term “fluorophore” as used herein refers to a molecule that absorbs light at a particular wavelength (excitation frequency) and subsequently emits light of a longer wavelength (emission frequency). The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety.

The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e., collisional) quenching or by Förster or fluorescence resonance energy transfer (“FRET”). Quenching by FRET is generally used in TaqMan® probes while proximal quenching is used in molecular beacon and Scorpion™ type probes.

In proximal quenching (a.k.a. “contact” or “collisional” quenching), the donor is in close proximity to the quencher moiety such that energy of the donor is transferred to the quencher, which dissipates the energy as heat as opposed to a fluorescence emission. In FRET quenching, the donor fluorophore transfers its energy to a quencher which releases the energy as fluorescence at a higher wavelength. Proximal quenching requires very close positioning of the donor and quencher moiety, while FRET quenching, also distance related, occurs over a greater distance (generally 1-10 nm, the energy transfer depending on R-6, where R is the distance between the donor and the acceptor). Thus, when FRET quenching is involved, the quenching moiety is an acceptor fluorophore that has an excitation frequency spectrum that overlaps with the donor emission frequency spectrum. When quenching by FRET is employed, the assay may detect an increase in donor fluorophore fluorescence resulting from increased distance between the donor and the quencher (acceptor fluorophore) or a decrease in acceptor fluorophore emission resulting from decreased distance between the donor and the quencher (acceptor fluorophore).

Suitable fluorescent moieties include the following fluorophores known in the art: 4-acetamido-4′-isothiocyanatostilbene-2,2′disulfonic acid, acridine and derivatives (acridine, acridine isothiocyanate) Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes), 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Black Hole Quencher™ (BHQ™) dyes (biosearch Technologies), BODIPY® R-6G, BOPIPY®530/550, BODIPY® FL, Brilliant Yellow, coumarin and derivatives (coumarin, 7-amino-4-methylcoumarin (AMC, Coumarin 120), 7-amino-4-trifluoromethylcouluarin (Coumarin 151)), Cy2®, Cy3®, Cy3®, Cy5®, Cy5.5®, cyanosine, 4′,6-diaminidino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin, diethylenetriamine pentaacetate, 4,4′-diisothiocyanatodihydro-stilbene-2,2′-disulfonic acid, 4,4′-diisothiocyanatostilbene-2,2′-disulfonic acid, 5-[dimethylamino]naphthalene-1-sulfonyl chloride (DNS, dansyl chloride), 4-(4′-dimethylaminophenylazo)benzoic acid (DABCYL), 4-dimethylaminophenylazophenyl-4′-isothiocyanate (DABITC), Eclipse™ (Epoch Biosciences Inc.), eosin and derivatives (eosin, eosin isothiocyanate), erythrosin and derivatives (erythrosin B, erythrosin isothiocyanate), ethidium, fluorescein and derivatives (5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET)), fluorescamine, IR144, IR1446, Malachite Green isothiocyanate, 4-methylumbelliferone, ortho cresolphthalein, nitrotyrosine, pararosaniline, Phenol Red, B-phycoerythrin, R-phycoerythrin, o-phthaldialdehyde, Oregon Green®, propidium iodide, pyrene and derivatives (pyrene, pyrene butyrate, succinimidyl 1-pyrene butyrate), QSY® 7, QSY® 9, QSY® 21, QSY® 35 (Molecular Probes), Reactive Red 4 (Cibacron® Brilliant Red 3B-A), rhodamine and derivatives (6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, sulforhodamine B, sulforhodamine 101, sulfonyl chloride derivative of sulforhodamine 101 (Texas Red)), N,N,N′,N′-tetramethyl-6-carboxyrhodamine (TAMRA), tetramethyl rhodamine, tetramethyl rhodamine isothiocyanate (TRITC), CAL Fluor Red 610, Quasar 670, riboflavin, rosolic acid, terbium chelate derivatives.

Other fluorescent nucleotide analogs can be used, see, e.g., Jameson, 278 Meth. Enzymol. 363-390 (1997); Zhu, 22 Nucl. Acids Res. 3418-3422 (1994). U.S. Pat. Nos. 5,652,099 and 6,268,132 also describe nucleoside analogs for incorporation into nucleic acids, e.g., DNA and/or RNA, or oligonucleotides, via either enzymatic or chemical synthesis to produce fluorescent oligonucleotides. U.S. Pat. No. 5,135,717 describes phthalocyanine and tetrabenztriazaporphyrin reagents for use as fluorescent labels.

The detectable label can be incorporated into, associated with or conjugated to a nucleic acid. Label can be attached by spacer arms of various lengths to reduce potential steric hindrance or impact on other useful or desired properties. See, e.g., Mansfield, 9 Mol. Cell. Probes 145-156 (1995). Detectable labels can be incorporated into nucleic acids by covalent or non-covalent means, e.g., by transcription, such as by random-primer labeling using Klenow polymerase, or nick translation, or amplification, or equivalent as is known in the art. For example, a nucleotide base is conjugated to a detectable moiety, such as a fluorescent dye, and then incorporated into nucleic acids during nucleic acid synthesis or amplification.

With Scorpion™ probes, sequence-specific priming and PCR product detection is achieved using a single molecule. The Scorpion™ probe maintains a stem-loop configuration in the unhybridized state. The fluorophore is attached to the 5′ end and is quenched by a moiety coupled to the 3′ end The 3′ portion of the stem also contains sequence that is complementary to the extension product of the primer. This sequence is linked to the 5′ end of a specific primer via a non-amplifiable monomer. After extension of the Scorpion™ primer, the specific probe sequence is able to bind to its complement within the extended amplicon thus opening up the hairpin loop. This prevents the fluorescence from being quenched and a signal is observed. A specific target is amplified by the reverse primer and the primer portion of the Scorpion™, resulting in an extension product. A fluorescent signal is generated due to the separation of the fluorophore from the quencher resulting from the binding of the probe element of the Scorpion™ to the extension product.

TaqMan® probes (Heid, et al., Genome Res 6: 986-994, 1996) use the fluorogenic 5′ exonuclease activity of Taq polymerase to measure the amount of target sequences in cDNA samples. TaqMan® probes are oligonucleotides that contain a donor fluorophore usually at or near the 5′ base, and a quenching moiety typically at or near the 3′ base. The quencher moiety may be a dye such as TAMRA or may be a non-fluorescent molecule such as 4-(4-dimethylaminophenylazo) benzoic acid (DABCYL). See Tyagi, et al., 16 Nature Biotechnology 49-53 (1998). When irradiated, the excited fluorescent donor transfers energy to the nearby quenching moiety by FRET rather than fluorescing. Thus, the close proximity of the donor and quencher prevents emission of donor fluorescence while the probe is intact.

TaqMan® probes are designed to anneal to an internal region of a PCR product. When the polymerase (e.g., reverse transcriptase) replicates a template on which a TaqMan® probe is bound, its 5′ exonuclease activity cleaves the probe. This ends the activity of the quencher (no FRET) and the donor fluorophore starts to emit fluorescence which increases in each cycle proportional to the rate of probe cleavage. Accumulation of PCR product is detected by monitoring the increase in fluorescence of the reporter dye (note that primers are not labeled). If the quencher is an acceptor fluorophore, then accumulation of PCR product can be detected by monitoring the decrease in fluorescence of the acceptor fluorophore.

In a suitable embodiment, real time PCR is performed using any suitable instrument capable of detecting fluorescence from one or more fluorescent labels. For example, real time detection on the instrument (e.g. a ABI Prism® 7900HT sequence detector) monitors fluorescence and calculates the measure of reporter signal, or Rn value, during each PCR cycle. The threshold cycle, or Ct value, is the cycle at which fluorescence intersects the threshold value. The threshold value is determined by the sequence detection system software or manually.

In one embodiment, the detection of the target nucleic acids can be accomplished by means of so called Invader™ technology (available from Third Wave Technologies Inc. Madison, Wis.). In this assay, a specific upstream “invader” oligonucleotide and a partially overlapping downstream probe together form a specific structure when bound to complementary DNA template. This structure is recognized and cut at a specific site by the Cleavase enzyme, and this results in the release of the 5′ flap of the probe oligonucleotide. This fragment then serves as the “invader” oligonucleotide with respect to synthetic secondary targets and secondary fluorescently labeled signal probes contained in the reaction mixture. This results in specific cleavage of the secondary signal probes by the Cleavase enzyme. Fluorescence signal is generated when this secondary probe, labeled with dye molecules capable of FRET, is cleaved. Cleavases have stringent requirements relative to the structure formed by the overlapping DNA sequences or flaps and can, therefore, be used to specifically detect single base pair mismatches immediately upstream of the cleavage site on the downstream DNA strand. See Ryan D et al. Molecular Diagnosis 4(2):135-144 (1999) and Lyamichev V et al. Nature Biotechnology 17:292-296 (1999), see also U.S. Pat. Nos. 5,846,717 and 6,001,567.

In some embodiments, melting curve analysis may be used to detect an amplification product. Melting curve analysis involves determining the melting temperature of an nucleic acid amplicon by exposing the amplicon to a temperature gradient and observing a detectable signal from a fluorophore. Melting curve analysis is based on the fact that a nucleic acid sequence melts at a characteristic temperature called the melting temperature (Tₘ) which is defined as the temperature at which half of the DNA duplexes have separated into single strands. The melting temperature of a DNA depends primarily upon its nucleotide composition. Thus, DNA molecules rich in G and C nucleotides have a higher Tₘ than those having an abundance of A and T nucleotides.

Where a fluorescent dye is used to determine the melting temperature of a nucleic acid in the method, the fluorescent dye may emit a signal that can be distinguished from a signal emitted by any other of the different fluorescent dyes that are used to label the oligonucleotides. In some embodiments, the fluorescent dye for determining the melting temperature of a nucleic acid may be excited by different wavelength energy than any other of the different fluorescent dyes that are used to label the oligonucleotides. In some embodiments, the second fluorescent dye for determining the melting temperature of the detected nucleic acid is an intercalating agent. Suitable intercalating agents may include, but are not limited to SYBR™ Green 1 dye, SYBR™ dyes, Pico Green, SYTO dyes, SYTOX dyes, ethidium bromide, ethidium homodimer-1, ethidium homodimer-2, ethidium derivatives, acridine, acridine orange, acridine derivatives, ethidium-acridine heterodimer, ethidium monoazide, propidium iodide, cyanine monomers, 7-aminoactinomycin D, YOYO-1, TOTO-1, YOYO-3, TOTO-3, POPO-1, BOBO-1, POPO-3, BOBO-3, LOLO-1, JOJO-1, cyanine dimers, YO-PRO-1, TO-PRO-1, YO-PRO-3, TO-PRO-3, TO-PRO-5, PO-PRO-1, BO-PRO-1, PO-PRO-3, BO-PRO-3, LO-PRO-1, JO-PRO-1, and mixture thereof. In suitable embodiments, the selected intercalating agent is SYBR™ Green 1 dye.

By detecting the temperature at which the fluorescence signal is lost, the melting temperature can be determined. In the disclosed methods, each of the amplified target nucleic acids may have different melting temperatures. For example, each of these amplified target nucleic acids may have a melting temperature that differs by at least about 1° C., more preferably by at least about 2° C., or even more preferably by at least about 4° C. from the melting temperature of any of the other amplified target nucleic acids. By observing differences in the melting temperature(s) of the EV, HSV-1/-2, and/or VZV targets from the respective amplification products, one can confirm the presence or absence of EV, HSV-1/-2, and/or VZV in the sample.

To minimize the potential for cross contamination, reagent and master mix preparation, specimen processing and PCR setup, and amplification and detection are all carried out in physically separated areas.

### Preparation of an Internal Control

As a quality control measure, an internal amplification control (IC) may be included in one or more samples to be extracted and amplified. The skilled artisan will understand that any detectable sequence that is not derived from EV, HSV-1/-2, or VZV can be used as the control sequence. These controls can be mixed with the sample (or with purified nucleic acids isolated from the sample), and amplified with sample nucleic acids using a pair complementary to the control sequence. If PCR amplification is successful, the internal amplification control amplicons can then be detected and differentiated from EV, HSV-1/-2, or VZV amplicons using a probe to the control sequence. Additionally, if included in the sample prior to purification of nucleic acids, the control sequences can also act as a positive purification control. In one embodiment, the internal amplification control comprises the QIPC nucleic acid, as described in U.S. patent application Ser. No. 11/830,759.

All publications, patent applications, issued patents, and other documents referred to in the present disclosure are herein incorporated by reference as if each individual publication, patent application, issued patent, or other document were specifically and individually indicated to be incorporated by reference in its entirety. Definitions that are contained in text incorporated by reference are excluded to the extent that they contradict definitions in this disclosure.

### EXAMPLES

The present invention is further illustrated by the following examples, which should not be construed as limiting in any way.

### Example 1

### Materials and Methods

In accordance with the methods of the present invention, viral pathogens were detected in biological samples following the procedures described in this example. Using methods similar to the ones described below, it would be possible for the skilled artisan to alter the parameters for the detection of additional target nucleic acids or use alternate probe/primer designs to the ones shown herein.

The diagnostic panel in this example is designed to detect any of Enterovirus (EV) RNA, Herpes Simplex virus type-1 & -2 (HSV-1/-2) DNA, or Varicella-Zoster virus (VZV) DNA in a single well reaction. Specifically, it is designed to detect Enterovirus RNA 5′ untranslated region (UTR), HSV-1 and -2 UL29 gene, and VZV gene 36. The assay includes reagents and primers for the simultaneous detection of nucleic acid from the target EV, HSV-1 and -2 and VZV. The assay may further include an internal control (IC) to verify adequate processing of the target viruses and to monitor the presence of inhibition in the amplification assay to avoid false negative results.

The diagnostic test involved below a two-step procedure: (1) the extraction of nucleic acid from the subject's CSF specimens for use as the testing template and (2) one-step RT-PCR using reverse transcription to convert target RNA to cDNA followed by the simultaneous amplification and detection of the target templates.

1. Materials

The diagnostic test used a master mix containing enzymes, buffers, and dNTPs assembled according to Table 5.

TABLE 5
Master Mix Composition

| | Master Mix | | Final Reaction | |
|---|---|---|---|---|
| Component | Concentration | | Concentration | |
| Tris-HCl, pH 8.3 | 100 | mM | 50 | nM |
| MgCl₂ | 5 | mM | 2.5 | mM |
| KCl | 20 | nM | 10 | nM |
| (NH₄)₂SO₄ | 10 | mM | 10 | mM |
| dNTPs (U, A, G, C) | 400 | μM | 200 | μM |
| FastStart DNA Polymerase (Roche) | 4 | U | 2 | U |

The diagnostic test used a primer mix comprising dye-labeled DQS Scorpion™ primers and reverse primers. The Scorpion™ primers and reverse primers were specific for each of the targets being detected, as well as for the IC template. Accordingly, the primer mix contained the following primers: a Scorpion™ primer for EV, a Scorpion™ primer for HSV-1/-2, a Scorpion™ primer for VZV a Scorpion™ primer for IC, a reverse primer for EV, a reverse primer for HSV-1 & -2, a reverse primer for VZV, and a reverse primer for IC. A summary of the targets and detectable labels associated with each Scorpion™ primer is shown in Table 6. A summary of the targets for the reverse primers is shown in Table 7. The final concentrations for the Scorpion™ primers were 400 nM for EV, 300 nM for HSV, 300 nM for VZV, and 100 nM for IC. The final concentrations of the reverse primers were 400 nM for EV, 300 nM for HSV, 300 nM for VZV, and 100 nM for IC.

TABLE 6
Labeled Scorpion ™ Primers

| | SEQ | | | | | |
|---|---|---|---|---|---|---|
| | ID | | | | | |
| Virus | NO: | Fluorophore | Absorbance | Emission | Target | Size |
| EV | 6 | FAM | 495 nm | 520 nm | 5′ UTR | 48 bp |
| HSV- | 11 | JOE | 520 nm | 548 nm | UL29 | 48 bp |
| 1/-2 | | | | | | |
| VZV | 15 | CAL Fluor | 590 nm | 610 nm | Gene 36 | 52 bp |
| | | Red 610 | | | | |
| IC | 17 | Quasar 670 | 647 nm | 667 nm | IC DNA | 37 bp |

TABLE 7
Reverse Primers

| Reverse Primer | SEQ ID NO: | Target | Size |
|---|---|---|---|
| EV | 8 | 5′ UTR | 22 bp |
| HSV-1/-2 | 10 | UL29 | 16 bp |
| VZV | 14 | Gene 36 | 23 bp |
| IC | 18 | IC DNA | 18 bp |

An IC Armored RNA fragment was included in each reaction to verify the success of the extraction procedure, monitor the quality of the reverse transcription and amplification reaction, and detect the presence of any amplification inhibitors. The IC comprises a Armored RNA fragment with a predetermined sequence cloned into a carrier vector. It was spiked into each specimen before nucleic acid extraction. The IC was constructed by Armored RNA technology (Asuragen, Tex.) and had a sequence according to SEQ ID NO:19:

(SEQ ID NO: 19)

TGTGATGGATATCTGCAGAATTCGCCCTTTGTTTCGACCTAGCTTGCCAG
TTCGCAGAATTTGTTGCTCGTCAGTCGTCGGCGGTTTTAAGGGCGAATTC
CAGCACACTGGCGGCCGTTA

Furthermore, a positive control (PC) reaction was run in a separate reaction well. The PC contained templates of PCR-amplified DNA fragments of the target regions for HSV (SEQ ID NO: 2) and VZV (SEQ ID NO: 3), and an Armored RNA particle containing the target region for EV (SEQ ID NO: 1).

The amplification reaction was prepared according to Table 8. The components were mixed and distributed to a optical 96-well plate. The plate was vortexed for 5 sec and centrifuged at 3000 rpm for 2 min.

TABLE 8
Amplification Reaction Mixture

| Component | Volume (μL) |
|---|---|
| Master Mix | 12.5 |
| Primer Mix | 2.5 |
| ImProm-II ™ Reverse Transcriptase | 0.5 |
| (Promega, Madison, WI) | |
| RNasin ® Plus RNase Inhibitor | 0.35 |
| (Promega, Madison WI) | |
| Patient Sample or Control | 5 |
| Water | to 25 μL |

2. Specimen Collection, Preparation, and Handling

Cerebrospinal fluid (CSF, 500 μl) was collected in a sterile container. Specimens were stored at 2° C.-8° C. or −20° C. until testing. Immediately before sample preparation, specimens were equilibrated to room temperature (15-25° C.). Next, 5 μl of the IC RNA at 300 copies/μl was spiked into 135 μL of each specimen. Nucleic acids were extracted using the QIAamp® Viral RNA Mini Kit (Cat. No. 52905, Qiagen, Valencia, Calif.). Extraction was according to the manufacturer's instructions except that the final elution used 40 μL of buffer AVE instead of the 200 μL recommended by the manufacturer. Nucleic acid samples were stored at −20° C., if not assayed immediately.

3. Nucleic Acid Amplification and Detection

The amplification reaction was performed using a ABI SDS 7500 Real-Time PCR Detection System with the following channel, dye, and analyte sets: Channel A: FAM for EV; Channel B: JOE for VZV; Channel D: CFR610 for HSV-1 and -2; Channel E: Q670 for IC; Calibration dye: none. The cycling parameters were as follows: 47° C. for 30 min; 95° C. for 10 min, and 45 cycles of 95° C. for 15 sect 60° C. for 45 sec. Following the run, the cutoffs was determined as in Table 9 below.

TABLE 9
Fluorescence Threshold and Cutoff Values

| | | Threshold | Positive | Equivocal | Negative |
|---|---|---|---|---|---|
| Analyte | Channel | (ΔRn) | cutoff (Ct) | zone (Ct) | cutoff (Ct) |
| EV | zFAM | 40000 | 42.0 | 42.1-44.0 | 44.1 |
| HSV | 610 | 50000 | 37.0 | 37.1-39.0 | 39.1 |
| VZV | JOE | 20000 | 40.0 | 40.1-42.0 | 42.1 |
| IC | 670 | 30000 | 40.0 | NA | NA |

4. Interpretation of Test Results

The Negative Control was the primary specimen matrix free of the target being detected or water. To verify the validity of the run, the negative control well should be reported as follows: FAM: Not detected; JOE: Not detected; CFR610: Not detected; Q670: Detected. If these results were not found, then the entire plate was interpreted as failed and was retested.

The Positive Control (PC) contained a DNA fragment of the target regions or an Armored RNA particle containing the target region. To verify the validity of the run, the PC was confirmed to be: FAM: Detected; JOE: Detected; CFR610: Detected; Q670: Detected. If these results were not found, then the entire plate was interpreted as failed, and was retested. Individual samples were interpreted based on the matrix shown in Table 10, where “+” is detected and “−” is not detected.

TABLE 10
Interpretation of Results

| | | Fluorophore/Target Result | | | |
|---|---|---|---|---|---|
| Test | | Q670 | FAM | JOE | CFR610 |
| Validity | Test Interpretation | IC | EV | VZV | HSV-1 & -2 |
| Retest | Retest patient sample | − | +/− | +/− | +/− |
| Valid | Patient sample is | + | − | − | − |
| | negative | | | | |
| Valid | EV is positive, | + | + | − | − |
| | HSV-1 & -2 is negative | | | | |
| | VZV is negative | | | | |
| Valid | EV is negative | + | − | + | − |
| | HSV-1 or -2 is positive | | | | |
| | rest | | | | |
| | VZV is negative | | | | |
| Valid | VZV is positive | + | − | − | + |
| | EV is negative | | | | |
| | HSV-1 & -2 is negative | | | | |
| Retest | Retest patient sample | +/− | + | + | − |
| Retest | Retest patient sample | +/− | + | − | + |
| Retest | Retest patient sample | +/− | − | + | + |
| Retest | Retest patient sample | +/− | + | + | + |

Results from these tests may be correlated with the clinical history, epidemiological data, and other data available to the attending physician in evaluating the patient. As with other diagnostic tests, negative results do not rule out the diagnosis of CNS infections by EV, HSV-1/-2, or VZV. For example, false negatives may occur when the infecting virus has genomic mutations, insertions, deletions, or rearrangements. Furthermore, false positive results may occur. Repeat testing or testing with a different device may be indicated in some settings, e.g. patients with a low likelihood of CNS infection by EV, HSV-1/-2, or VZV.

### Example 2

### Enterovirus Serotype Detection Coverage

A comparison of the genomic sequences from EV serotypes demonstrated that the EV RNA genome is highly polymorphic and there is no identical sequence among all of the serotypes. However, the 5′-UTR contains three very small, but highly conserved regions across most classified EV serotypes (See U.S. Pat. No. 5,075,212). Although it is highly conserved, this region is not a 100% consensus for all EV serotypes. Various mutations have been identified in the region, and mutations in the region could affect the PCR amplification and detection. EV serotypes that contain mutations in the 5′-UTR conserved region were identified (indicated by the * in Table 11). These serotypes were defined as “variable serotypes”.

To verify the serotype detection coverage for 64 EV serotypes, each serotype was assayed using the methods described in Example 1 (herein “Simplexa™”). All ATCC strains of the “EV serotype panel” were extracted by QIAmp® Viral RNA Mini Kit (Qiagen Cat. No. 52906). The extracted RNA was diluted 100- and 1000-fold in TE for testing. An identical sample panel was tested by singleplex TaqMan® assay, according to ABI's recommended procedure (Cat. No. 4352042). The results comparing the detection capability of Simplexa™ and the TaqMan® assay are shown in Table 11.

TABLE 11
Detection of EV Serotypes

| | CDC | CDC | ATCC | | TaqMan ® | Simplexa ™ |
|---|---|---|---|---|---|---|
| Serotype | Rank | % | ID | Dilution | (Ct) | (Ct) |
| EC9* | 1 | 11.8 | VR 39 | 1/100 | 22.69 | 21.51 |
| EC11 | 2 | 11.4 | VR 41 | 1/100 | 29.35 | 22.66** |
| EC19 | 35 | 0.2 | VR 49 | 1/100 | 26.96 | 27.08 |
| EC23 | 39 | 0.1 | VR 53 | 1/100 | ND | ND |
| CVA11 | 54 | 0.1 | VR 169 | 1/100 | 21.00 | 22.17 |
| CVA19* | NA | NA | VR 177 | 1/100 | 20.91 | 22.19 |
| CVA4 | 29 | 0.4 | VR 184 | 1/100 | 23.05 | 22.83 |
| EV68 | 47 | 0.1 | VR 561 | 1/100 | 29.64 | 29.47 |
| CVA24* | 41 | 0.1 | VR 583 | 1/100 | 21.09 | 21.13 |
| EV71 | 27 | 0.5 | VR 784 | 1/100 | 24.19 | 23.29 |
| EV70 | 58 | 0.1 | VR 836 | 1/100 | 22.09 | 22.00 |
| CVA5 | 37 | 0.1 | VR | 1/100 | 22.75 | 23.39 |
| | | | 1010 | | | |
| CVA17* | 55 | 0.1 | VR | 1/100 | 23.77 | 25.13 |
| | | | 1023 | | | |
| EC1 | 30 | 0.4 | VR | 1/100 | 31.28 | 27.14 |
| | | | 1038 | | | |
| EC22 | 16 | 1.8 | VR | 1/100 | ND | ND |
| | | | 1063 | | | |
| EC25 | 20 | 1.1 | VR | 1/100 | 35.70 | 27.24** |
| | | | 1066 | | | |
| EC26 | 52 | 0.1 | VR | 1/100 | 35.57 | 25.90** |
| | | | 1067 | | | |
| EC29 | 46 | 0.1 | VR | 1/100 | 24.24 | 24.24 |
| | | | 1070 | | | |
| EC31 | 25 | 0.7 | VR | 1/100 | 29.76 | 20.09 |
| | | | 1073 | | | |
| EC12 | 42 | 0.1 | VR | 1/100 | 21.34 | 21.59 |
| | | | 1563 | | | |
| EC9* | 1 | 11.8 | VR 39 | 1/1000 | 26.95 | 27.22 |
| EC11 | 2 | 11.4 | VR 41 | 1/1000 | 33.87 | 28.20** |
| EC19 | 35 | 0.2 | VR 49 | 1/1000 | 30.76 | 33.07 |
| EC23 | 39 | 0.1 | VR53 | 1/1000 | ND | ND |
| CVA11 | 54 | 0.1 | VR 169 | 1/1000 | 24.59 | 25.46 |
| CVA19* | NA | NA | VR 177 | 1/1000 | 25.18 | 27.54 |
| CVA4 | 29 | 0.4 | VR 184 | 1/1000 | 27.11 | 27.95 |
| EV68 | 47 | 0.1 | VR 561 | 1/1000 | 33.97 | 35.22 |
| CVA24* | 41 | 0.1 | VR 583 | 1/1000 | 25.41 | 27.59 |
| EV71 | 27 | 0.5 | VR 784 | 1/1000 | 26.36 | 31.02 |
| EV70 | 58 | 0.1 | VR 836 | 1/1000 | 28.11 | 27.93 |
| CVA5 | 37 | 0.1 | VR | 1/1000 | 26.54 | 28.74 |
| | | | 1010 | | | |
| CVA17* | 55 | 0.1 | VR | 1/1000 | 27.24 | 28.07 |
| | | | 1023 | | | |
| EC1 | 30 | 0.4 | VR | 1/1000 | 34.85 | 33.38 |
| | | | 1038 | | | |
| EC22 | 16 | 1.8 | VR | 1/1000 | ND | ND |
| | | | 1063 | | | |
| EC25 | 20 | 1.1 | VR | 1/1000 | 43.24 | 33.88** |
| | | | 1066 | | | |
| EC26 | 52 | 0.1 | VR | 1/1000 | 38.46 | 32.44** |
| | | | 1067 | | | |
| EC29 | 46 | 0.1 | VR | 1/1000 | 28.43 | 31.34 |
| | | | 1070 | | | |
| EC31 | 25 | 0.7 | VR | 1/1000 | 34.10 | 27.25** |
| | | | 1073 | | | |
| EC12 | 42 | 0.1 | VR | 1/1000 | 25.13 | 26.95 |
| | | | 1563 | | | |

Ct = Cycle threshold;
ND = Ct value was undetermined;
*non-variable serotypes;
**significant earlier Ct detection was observed for Simplexa ™ assay vs. TaqMan ® assay.

In this study, the EV serotype panel was tested by the Simplexa™ Viral Encephalitis assay and singleplex EV TaqMan® assay. The panel included all ATCC variable serotypes. The panel also included a few serotypes which are not variable, including EC9, the most prevalent serotype on the CDC's list. By combining all variable and non-variable strains in the panel, all known “genotypes” of EV have been incorporated. Therefore, this EV serotype panel is considered to represent all serotypes with known sequences. All strains in the panel were collected from ATCC pretyped viral culture stock. The serotype EC6 was not available from ATCC. The Simplexa™ assay detected all serotypes except EC22 and EC23 (renamed Human Parechovirus 1 and 2 on CDC's list; also not detected by TaqMan). Accordingly, compared with singleplex TaqMan® assay, most of the variable serotypes were detected with parallel Ct values.

For EC11, EC25, EC26 and EC31, the Simplex™ assay showed significantly lower Ct's (5-10) compared with the TaqMan® assays which indicated improved detection sensitivity of Simplexa™ on some of major variable serotypes. Among these serotypes, EV11 was ranked number 2 on CDC surveillance data with 11.4% of all reported cases.

This study verified that the Simplexa™ assay is able to detect all EV classified serotypes (excluding: EV6 which was not available from ATCC, and EC22 and EC23 which were on the CDC's list but reclassified). This study also verified that the Simplexa™ assay performed equally well to, or better than, singleplex TaqMan® EV assay in EV serotype detection coverage. As such, the methods of the present invention are useful in diagnostic assays for the detection of EV viral pathogens.

### Example 3

### Sensitivity and Specificity of the Detection Methods of the Invention

The sensitivity and specificity of the detection methods of the invention (herein “Simplexa™”) were investigated in this Example. A total of 350 CSF specimens were collected from the Focus Reference Lab. These specimens were originally submitted for Enterovirus (EV), Herpes Simplex virus type 1- and/or -2 (HSV-1/-2) and/or Varicella Zostar Virus (VZV) molecular tests. The majority of the samples were collected during 2004 and stored at −80° C. prior to extraction.

Due to the limited number of VZV specimens, 22 spiked VZV samples were contrived. VZV viral strain (Strain Ellen Lot 24W) and commercial CSF matrix (Medical Analysis System, Inc. Lot SF07051) were used to create the spiked samples. The spiked samples were made to cover a wide range of viral CSF viral loads with a 2-fold dilution series including 22 samples, starting from 100× ATCC stock dilution. The lower end of the concentration was made to reach and/or cross beyond the cutoff area, or to create a discrepancy. No spiked samples were made for EV or HSV.

Specimens were extracted and amplification was performed as described in Example 1. Singleplex TaqMan® assays for HSV, VZV, and EV were used as a comparison. For HSV and VZV, the discrepant samples between Simplexa™ and singleplex TaqMan® and specimens surrounding the cutoff region were re-evaluated by re-extraction and retest in duplicates. The interpretation of data was determined by the rule of majority (equal or greater than two-thirds of 2× retest plus 1× first test). Repeated tests were used to establish the cutoff, but not used in calculating or correcting the sensitivity and specificity values. For EV, discrepant samples were reevaluated by using a 2-step TaqMan® assay. The discrepant samples were re-extracted and retested. For EV discrepant samples, the result interpretation was determined by re-extraction and retest only.

The cutoff and equivocal zones were determined by comparing Simplexa™ and singleplex TaqMan® results using the visual receiver-operator characteristics (ROC) approach (Jacobson R. H., Manual of Standards for Diagnostic Tests and Vaccines, p. 8-15, 1996). The cutoff and equivocal zone for each analyte was determined independently.

A sample with a Ct value falling in the equivocal zone was determined and interpreted in the following way: (1) the specimen was rextracted and retested by Simplexa™ assay twice, and (2) the two retest results were combined with the first test result, and a call was made based on majority rules or majority average rules. A result occurring in equal or greater than ⅔ of the tests determined the interpretation by majority rules. One positive/one equivocal/one negative was be interpreted as equivocal by the majority average rules.

As described above, the sensitivity and specificity of the EV, HSV, and VZV assays was compared between Simplexa™ and singleplex TaqMan®. For calculating HSV and VZV sensitivity and specificity, only first test results were used. For calculating EV sensitivity and specificity, only single test results were used (311 from the first extract and 31 from second extract). Results from repeated tests of discrepant samples and correction (if applied) outside the equivocal zone were not used. Therefore, the numbers were calculated in most conservative way and reflected the original single test results. Overall concordance (test to test) was 98.4% (1025/1042). The sensitivity of Simplexa™ assay was 93.33% for HSV, 92.31% for VZV, and 97.62% for EV. The specificity of Simplexa™ Viral Encephalitis assay was 99.14% for HSV, 98.57% for VZV, and 97.37% for EV.

Based on 350 samples, the CSF pathogen “load” distribution was analyzed. In the panel, samples positive for EV had a Ct range of 28-42; samples positive for HSV-1/-2 had a Ct range of 21-37; and samples positive for VZV had a Ct range of 20-40. However, since majority of the specimens were 3 years old or more, the distribution value may not reflect the true value of first test from fresh specimens, especially for EV.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

What is claimed is:

1. A method for identifying the presence or absence of a viral pathogen in a biological sample, comprising assaying the sample for one or both of:
(a) a HSV UL29 nucleic acid or a fragment thereof; and
(b) a VZV gene 36 nucleic acid or fragment thereof;
wherein the presence of one or both of the nucleic acids or fragments specified in (a) and (b) indicates that the biological sample contains the viral pathogen associated with said nucleic acids or fragments.

2. The method of claim 1 further comprising assaying the sample for:
(c) an enterovirus 5′ UTR nucleic acid or a fragment thereof, wherein the presence of the nucleic acid or fragment specified in (c) indicates that the biological sample contains enterovirus.

3. The method of claim 2 comprising assaying the sample for (a) and (c).

4. The method of claim 3, wherein the step of assaying comprises
(a) contacting the biological sample with one or more primers suitable for amplifying an enterovirus 5′ UTR nucleic acid or fragment thereof; and one or more primers suitable for amplifying an HSV UL29 nucleic acid or a fragment thereof;
(b) performing a multiplex amplification reaction comprising the primers of step (a) under conditions suitable to produce a first reaction product when the enterovirus 5′ UTR gene is present in said sample, and a second reaction product when HSV UL29 nucleic acid is present in said sample; and
(c) detecting the presence one or both of the first and second reaction products.

5. The method of claim 2 comprising assaying the sample for (a), (b), and (c).

6. The method of claim 5, wherein the step of assaying comprises
(a) contacting the biological sample with one or more primers suitable for amplifying an enterovirus 5′ UTR nucleic acid or fragment thereof; one or more primers suitable for amplifying an HSV UL29 nucleic acid or a fragment thereof; and one or more primers suitable for amplifying a VZV gene 36 nucleic acid or a fragment thereof;
(b) performing a multiplex amplification reaction comprising the primers of step (a) under conditions suitable to produce a first reaction product when the enterovirus 5′ UTR gene is present in said sample, a second reaction product when HSV UL29 nucleic acid is present in said sample; a third reaction product suitable for amplifying the VZV gene 36 nucleic acid is present in said sample; and
(c) detecting the presence of one or more of the first, second, or third reaction products.

7. The method of claim 6, wherein the first reaction product has at least 30 contiguous nucleotides from the sequence of SEQ ID NO: 1.

8. The method of claim 6, wherein at least one primer suitable for amplifying an enterovirus 5′ UTR nucleic acid or fragment thereof is selected from the group consisting of: SEQ ID NOS: 4, 8, and complements thereof.

9. The method of claim 6, wherein the first reaction product is detected using a probe comprising a fluorescent label.

10. The method of claim 9, wherein the probe and a primer suitable for amplifying an enterovirus 5′ UTR nucleic acid or fragment thereof are part of a bi-functional molecule.

11. The method of claim 10, wherein the bi-functional molecule has a sequence selected from the group consisting of: the constructs of SEQ ID NOS: 6 & 4 and 7 & 4 and complements thereof.

12. The method of claim 6, wherein the second reaction product has at least 30 contiguous nucleotides from the sequence of SEQ ID NO: 2.

13. The method of claim 6, wherein at least one primer suitable for amplifying an HSV UL29 nucleic acid or a fragment thereof is selected from the group consisting of: SEQ ID NOS: 9-10, and complements thereof.

14. The method of claim 6, wherein the second reaction product is detected using a probe comprising a fluorescent label.

15. The method of claim 14, wherein the probe and a primer suitable for amplifying an HSV UL29 nucleic acid or a fragment thereof are part of a bi-functional molecule.

16. The method of claim 15, wherein the bi-functional molecule has a sequence according to the construct of SEQ ID NO: 11 & 9 or a complement thereof.

17. The method of claim 6, wherein the third reaction product has at least 30 contiguous nucleotides from the sequence of SEQ ID NO: 3.

18. The method of claim 6, wherein at least one primer suitable for amplifying a VZV gene 36 nucleic acid or a fragment thereof is selected from the group consisting of: SEQ ID NOS: 13-14, and complements thereof.

19. The method of claim 6, wherein the third reaction product is detected using a probe comprising a fluorescent label.

20. The method of claim 19, wherein the probe and a primer suitable for amplifying a VZV gene 36 nucleic acid or a fragment thereof are part of a bi-functional molecule.

21. The method of claim 20, wherein the bi-functional molecule has a sequence according to the construct of SEQ ID NO: 15 & 13 or a complement thereof.

22. The method of claim 2, wherein the step of detecting comprises performing an invasive cleavage assay on one or more of the genes or fragments specified in (a), (b), or (c).

23. A method of diagnosing a subject for infection with a viral pathogen, comprising assaying a biological sample from the subject for the presence or absence of one or both of:
(b) a HSV UL29 nucleic acid or a fragment thereof,
(c) a VZV gene 36 nucleic acid or a fragment thereof,
wherein the presence of said nucleic acids or fragments indicates that the individual is affected with the viral pathogen associated with said nucleic acids or fragments.

24. The method of claim 23 comprising assaying a biological sample for (c) an enterovirus 5′ UTR nucleic acid or a fragment thereof, wherein the presence of said nucleic acid or fragment indicates that the individual is affected with enterovirus.

25. The method of claim 24, wherein said method comprises amplifying each of said enterovirus 5′ UTR nucleic acid or a fragment thereof, HSV UL29 nucleic acid or a fragment thereof, and VZV gene 36 nucleic acid or a fragment thereof.

26. The method of claim 24, wherein the step of assaying comprises
(a) contacting the biological sample with one or more primers suitable for amplifying an enterovirus 5′ UTR nucleic acid or fragment thereof; one or more primers suitable for amplifying an HSV UL29 nucleic acid or a fragment thereof; and one or more primers suitable for amplifying a VZV gene 36 nucleic acid or a fragment thereof;
(b) performing a multiplex amplification reaction comprising the primers of step (a) under conditions suitable to produce a first reaction product when the enterovirus 5′ UTR nucleic acid is present in said sample, a second reaction product when HSV UL29 nucleic acid is present in said sample; a third reaction product suitable for amplifying the VZV gene 36 nucleic acid is present in said sample; and
(c) detecting the presence or absence of one or more of the first, second, or third reaction products.

27. The method of claim 26, wherein the first reaction product has at least 30 contiguous nucleotides from the sequence of SEQ ID NO: 1.

28. The method of claim 26, wherein at least one primer suitable for amplifying an enterovirus 5′ UTR nucleic acid or fragment thereof is selected from the group consisting of: SEQ ID NOS: 4, 8, and complements thereof.

29. The method of claim 26, wherein the first reaction product is detected using a probe comprising a fluorescent label.

30. The method of claim 29, wherein the probe and a primer suitable for amplifying an enterovirus 5′ UTR nucleic acid or fragment thereof are part of a bi-functional molecule.

31. The method of claim 30, wherein the bi-functional molecule has a sequence selected from the group consisting of: the constructs of SEQ ID NOS: 6 & 4 and 7 & 4 and complements thereof.

32. The method of claim 26, wherein the second reaction product has at least 30 contiguous nucleotides from the sequence of SEQ ID NO: 2.

33. The method of claim 26, wherein at least one primer suitable for amplifying an HSV UL29 nucleic acid or a fragment thereof is selected from the group consisting of: SEQ ID NOS: 9-10, and complements thereof.

34. The method of claim 26, wherein the second reaction product is detected using a probe comprising a fluorescent label.

35. The method of claim 34, wherein the probe and a primer suitable for amplifying an HSV UL29 nucleic acid or a fragment thereof are part of a bi-functional molecule.

36. The method of claim 35, wherein the bi-functional molecule has a sequence according to the construct of SEQ ID NO: 11 & 9 or a complement thereof.

37. The method of claim 26, wherein the third reaction product has at least 30 contiguous nucleotides from the sequence of SEQ ID NO: 3.

38. The method of claim 26, wherein at least one primer suitable for amplifying a VZV gene 36 nucleic acid or a fragment thereof is selected from the group consisting of: SEQ ID NOS: 13-14, and complements thereof.

39. The method of claim 26, wherein the third reaction product is detected using a probe comprising a fluorescent label.

40. The method of claim 39, wherein the probe and a primer suitable for amplifying a VZV gene 36 nucleic acid or a fragment thereof are part of a bi-functional molecule.

41. The method of claim 40, wherein the bi-functional molecule has a sequence according to the construct of SEQ ID NO: 15 & 13 or a complement thereof.

42. The method of claim 26, wherein the method comprises real-time PCR.

43. The method of claim 26, wherein the step of detecting comprises performing an invasive cleavage assay on one or more of the nucleic acids or fragments specified in (a), (b), or (c).

44. A kit comprising one or more of the primer pairs selected from the group consisting of:
a first primer pair suitable for amplifying an HSV UL29 nucleic acid or a fragment thereof and a probe capable of specifically hybridizing to the HSV UL29 nucleic acid; and
a second primer pair suitable for amplifying a VZV gene 36 nucleic acid or a fragment thereof and a probe capable of specifically hybridizing to the VZV gene 36 nucleic acid.

45. The kit of claim 44 comprising a third primer pair suitable for amplifying an enterovirus 5′ UTR nucleic acid or fragment thereof and a probe capable of specifically hybridizing to the enterovirus 5′ UTR nucleic acid.

46. The kit of claim 45, wherein the primer pair suitable for amplifying an enterovirus 5′ UTR nucleic acid or fragment thereof specifically hybridizes to a nucleic acid having the sequence of SEQ ID NO: 1.

47. The kit of claim 45, wherein at least one primer of the third primer pair comprises a sequence selected from the group consisting of: SEQ ID NOS: 4, 8, and complements thereof.

48. The kit of claim 44, wherein the primer pair suitable for amplifying an HSV UL29 nucleic acid or fragment thereof specifically hybridizes to a nucleic acid of SEQ ID NO: 2.

49. The kit of claim 44, wherein the primer pair suitable for amplifying a VZV gene 36 nucleic acid or fragment thereof specifically hybridizes to a nucleic acid of SEQ ID NO: 3.

50. The kit of claim 44, wherein at least one primer of the first primer pair comprises a sequence selected from the group consisting of: SEQ ID NOS: 9-10, and complements thereof.

51. The kit of claim 44, wherein at least one primer of the second primer pair comprises a sequence selected from the group consisting of: SEQ ID NOS: 13-14, and complements thereof.


SEQUENCE LISTING (19 sequences)

SEQ ID NO: 1 - DNA, 232 bp, Artificial Sequence
  Description of Artificial Sequence Synthetic polynucleotide
ttggtagtcctccggcccctgaatgcggctaatcctaactgcggagcacgtgcccacaaaccagtgggtagtgtgtcgtaacgggcaactctgcagcggaaccgactactttgggtgtccgtgtttccttttattcttatactggctgcttatggtgacaattgagagattgttaccatatagctattggattggccatccggtgactaacagagctattatatacctgttt

SEQ ID NO: 2 - DNA, 120 bp, Herpes simplex virus
ccgcgtggaactgcttcagcagaaagcccagcggtccgaggaggatgtccacgcgcttgtcgggcttctggtaggcgctctggaggctggcgacccgcgccttggcggcctcggacgcgt

SEQ ID NO: 3 - DNA, 179 bp, Varicella-Zoster virus
tttcccttgtccagatacttagtgggagatatgtccccagcggcgcttcctgggttattgtttacgcttccgctgaaccccccgggaccaacttggtagtttgtaccgtttcactccccagtcatttatccagagtaagcaaacgggccagaccgggagaaacggttaatctgccgttt

SEQ ID NO: 4 - DNA, 16 bp, Artificial Sequence
  Description of Artificial Sequence Synthetic oligonucleotide
tccggcccctgaatgc

SEQ ID NO: 5 - DNA, 33 bp, Artificial Sequence
  Description of Artificial Sequence Synthetic oligonucleotide
agcgcgcacccaaagtagtcggttccgcgcgct

SEQ ID NO: 6 - DNA, 32 bp, Artificial Sequence
  Description of Artificial Sequence Synthetic oligonucleotide
agcggcacggacacccaaagtagtcggccgct

SEQ ID NO: 7 - DNA, 30 bp, Artificial Sequence
  Description of Artificial Sequence Synthetic oligonucleotide
agcgggccaaagtagtcggttccgcccgct

SEQ ID NO: 8 - DNA, 22 bp, Artificial Sequence
  Description of Artificial Sequence Synthetic oligonucleotide
caattgtcaccataagcagcca

SEQ ID NO: 9 - DNA, 18 bp, Artificial Sequence
  Description of Artificial Sequence Synthetic oligonucleotide
ggtccgaggaggatgtcc

SEQ ID NO: 10 - DNA, 16 bp, Artificial Sequence
  Description of Artificial Sequence Synthetic oligonucleotide
cgtccgaggccgccaa

SEQ ID NO: 11 - DNA, 26 bp, Artificial Sequence
  Description of Artificial Sequence Synthetic oligonucleotide
agcgctgagcgcctaccagaagcgct

SEQ ID NO: 12 - DNA, 30 bp, Artificial Sequence
  Description of Artificial Sequence Synthetic oligonucleotide
aggcgcctaccagaagcccgacaagcgcct

SEQ ID NO: 13 - DNA, 25 bp, Artificial Sequence
  Description of Artificial Sequence Synthetic oligonucleotide
gttattgtttacgcttcccgctgaa

SEQ ID NO: 14 - DNA, 23 bp, Artificial Sequence
  Description of Artificial Sequence Synthetic oligonucleotide
gcccgtttgcttactctggataa

SEQ ID NO: 15 - DNA, 28 bp, Artificial Sequence
  Description of Artificial Sequence Synthetic oligonucleotide
agcggagtgaaacggtacaaactccgct

SEQ ID NO: 16 - DNA, 22 bp, Artificial Sequence
  Description of Artificial Sequence Synthetic oligonucleotide
attcgccctttgtttcgaccta

SEQ ID NO: 17 - DNA, 16 bp, Artificial Sequence
  Description of Artificial Sequence Synthetic oligonucleotide
tgcgaactggcaagct

SEQ ID NO: 18 - DNA, 18 bp, Artificial Sequence
  Description of Artificial Sequence Synthetic oligonucleotide
ccgacgactgacgagcaa

SEQ ID NO: 19 - DNA, 120 bp, Artificial Sequence
  Description of Artificial Sequence Synthetic polynucleotide
tgtgatggatatctgcagaattcgccctttgtttcgacctagcttgccagttcgcagaatttgttgctcgtcagtcgtcggcggttttaagggcgaattccagcacactggcggccgtta